(19) United States
(12) Patent Application Publication
(10) Pub. No.: US 2007/0270493 A1
(43) Pub. Date: Nov. 22, 2007

(54) Composition with Preventive or Improvement Effect on Stress-Induced Brain Function Impairment and Related Symptoms or Diseases

(76) Inventors: Manabu Sakakibara, Shizuoka (JP); Yoshiyuki Ishikura, Osaka (JP)

Correspondence Address: DRINKER BIDDLE & REATH (DC), 1500 K STREET, N.W., SUITE 1100, WASHINGTON, DC, 20005-1209, US

(21) Appl. No.: 11/662,477

(22) PCT Filed: Mar. 18, 2005
(86) PCT No.: PCT/JP05/05622

§ 371 Date: Mar. 12, 2007

(30) Foreign Application Priority Data
Sep. 17, 2004 (JP) 2004-271958

(51) Int. Cl. A61K 31/202; A61P 25/00; A61P 25/24

(52) U.S. Cl. 514/560

(57) ABSTRACT

A composition with a preventive or improvement effect on stress-induced brain function impairment and related symptoms or diseases, comprising arachidonic acid and/or a compound comprising arachidonic acid as a constituent fatty acid.

### TECHNICAL FIELD OF THE INVENTION

The present invention relates to a preventive or improvement agent for stress-induced brain function impairment and related symptoms or diseases, comprising as an active ingredient arachidonic acid and/or a compound comprising arachidonic acid as a constituent fatty acid, as well as to a composition with a preventive or improvement effect on stress-induced brain function impairment and related symptoms or diseases, and a method for its production. More specifically, the invention relates to a preventive or improvement agent for stress-induced memory and learning ability impairment, emotional disorders (such as depression) and the like, comprising as an active ingredient at least one selected from the group consisting of arachidonic acid, arachidonic acid alcohol esters, and triglycerides, phospholipids or glycolipids wherein all or a portion of the constituent fatty acid is arachidonic acid, as well as to a composition with such a preventive or improvement effect and a method for its production.

### BACKGROUND ART

Stress is recognized as a response which can lead to brain disorders. After a recorded event in which the death of apes resulted from overcrowding stress during long-distance transport fifteen years ago, the dead apes were examined and found to have signs of serious stress, including gastric ulcers, immunodeficiency and hypertrophic adrenal glands, while exfoliation of pyramidal cells of the CA3 region of the hippocampus was also reported (J. Neurosci. 9, 1705, 1989). Since publishing of this report, researchers began to focus on the psychological causes of brain disorders, and in particular, advances have been made in research on brain function impairment caused by stress.

Highly frequent stimulation of the brain hippocampus is known to lead to a phenomenon which includes synapse excitation and subsequent highly persistent synapse response. This is known as hippocampal LTP (long-term potentiation), a result of synaptic plasticity and one of the indicators for brain function evaluation. M. A. Lynch et al. reported that the hippocampal LTP of rats subjected to mild stress induced by separately breeding is demonstrably reduced compared to group-housed controls (J. Neurosci. 18, 2974, 1998). Thus, stress clearly contributes to brain function impairment.

Blood cortisol levels increase during periods of stress, and McEwen et al. have reported that Type 1 glucocorticoid receptors function in the hippocampus under physiological conditions, while Type 2 glucocorticoid receptors are active during times of corticosterone increase by stress; Type 1 receptors are protective in the hippocampal dentate gyrus, whereas Type 2 receptors tend to exacerbate neuropathy (Ann. NY Acad. Sci. 512, 394, 1987). Recently, increased blood IL-1β has been reported in post-traumatic stress disorder patients (Biol. Psychiatry 42, 345, 1997), and as the relationship between IL-1β and neuropathy has attracted researcher's attention, the possibility has been suggested that glucocorticoid receptor-mediated IL-1β increase in the hippocampus may contribute to neuropathy; however, much still remains unknown at the current time.

Research and development are also progressing in the area of discovering agents effective for the treatment of brain disorders (cerebral circulation/metabolism enhancers, anti-dementia drugs, etc.). Specifically, studies have focused on methods of improving brain energy metabolism through more efficient neuronal absorption of nutrients for activation of cellular function (increasing intracerebral glucose, for example), methods of improving brain circulation with the aim of more adequately providing the necessary nutrients and oxygen to neurons (cerebral blood flow enhancement methods, for example), methods of activating neurotransmission at the synaptic cleft by neurotransmitters (providing neurotransmitter precursors (for example, choline or acetyl CoA supplementation), inhibiting conversion of released neurotransmitters (for example, acetylcholinesterase inhibition), increasing neurotransmitter release (for example, augmentation of acetylcholine or glutamate release), and activating neurotransmitter receptors), or methods of protecting neurocyte membranes (for example, antioxidant treatment, membrane component supplementation or anti-atherosclerotic treatment). To date, however, no satisfactorily effective therapeutic agent has been discovered.

It has also become apparent that the pharmacological mechanism by which conventional drugs are efficacious for treatment of brain function is distinct from the pharmacological mechanism of stress-related brain function impairment, for which reason, presumably, the conventional agents by themselves have not been effective for prevention or improvement of stress-induced brain function impairment.

The progression of stress-related brain function impairment can be slowed by removing the cause of stress, and this is one obvious course for prevention and improvement; however, such a method is difficult to realize given the stressful nature of modern society. Thus, absolutely no drug has existed which is safe enough to be readily administered even to infants or the elderly, and which has a preventive or improvement effect on stress-related brain function impairment and its associated symptoms or diseases.

The brain consists of a lipid mass-like tissue, with phospholipids constituting about ⅓ of the white matter and about ¼ of the gray matter. The polyunsaturated fatty acids in phospholipids of the various cell membranes in the brain consist primarily of arachidonic acid and docosahexaenoic acid. However, arachidonic acid and docosahexaenoic acid cannot be synthesized de novo in animal bodies and must be directly or indirectly obtained through diet (for example, as the arachidonic acid and docosahexaenoic acid precursors, linoleic acid and α-linolenic acid). Consequently, while it has been supposed that arachidonic acid plays an important role in maintaining cerebral function, this has not been concretely substantiated because of a lack of adequate sources of arachidonic acid.

Several inventions have been disclosed which utilize arachidonic acid for maintenance of brain function. In Japanese Unexamined Patent Publication HEI No. 10-101568, “Brain function improvement and nutritive composition”, there is disclosed a ganglioside and arachidonic acid combination, as a means for providing a novel brain function improvement agent and a nutritive composition comprising it. Also, Japanese Unexamined Patent Publication No. 2003-048831, “Composition with preventive or improvement effect on symptoms and diseases associated with brain function impairment”, describes as test examples experiments wherein brain function decline in aged rats is improved by arachidonic acid. Still, these inventions are based on the conventional mode of improving brain function, whereas nothing is indicated regarding an effect of arachidonic acid against stress-induced brain function impairment.

Patent document 1: Japanese Unexamined Patent Publication HEI No. 10-101568

Patent document 2: Japanese Unexamined Patent Publication No. 2003-048831

Non-patent document 1: J. Neurosci. 9, 1705, 1989

Non-patent document 2: J. Neurosci. 18, 2974, 1998

Non-patent document 3: Ann. NY Acad. Sci. 512, 394, 1987

Non-patent document 4: Biol. Psychiatry 42, 345, 1997

### DISCLOSURE OF THE INVENTION

Thus, a strong demand exists for development of pharmaceuticals which prevent and improve stress-induced brain function impairment and its related symptoms or diseases, as well as such compounds which are highly suitable for consumption and lacking notable side effects.

As a result of much diligent research conducted with the purpose of elucidating the preventive or improvement effects on stress-induced brain function impairment and its associated symptoms and diseases by agents comprising as active ingredients arachidonic acid and/or compounds including arachidonic acid as a constituent fatty acid, the present inventors found, surprisingly, that the active ingredients of the invention exhibit apparent behavioral pharmacologic effects in mice subjected to restraint stress and evaluated by a Morris water maze learning test.

We also succeeded in realizing industrial production of a triglyceride containing at least 10% microorganism-generated arachidonic acid, and supplied the triglyceride for testing in order to elucidate the effect of the invention.

Specifically, the present invention provides a preventive or improvement agent for stress-induced brain function impairment and related symptoms or diseases and a composition with a preventive or improvement effect on stress-induced brain function impairment and related symptoms or diseases, comprising as an active ingredient arachidonic acid and/or a compound comprising arachidonic acid as a constituent fatty acid, as well as a method for their production.

More specifically, the invention provides a preventive or improvement agent for stress-induced memory and learning ability impairment or emotional disorders (such as depression or melancholia), comprising as an active ingredient at least one selected from the group consisting of arachidonic acid, arachidonic acid alcohol esters, and triglycerides, phospholipids or glycolipids wherein all or a portion of the constituent fatty acid is arachidonic acid, as well as to a composition with such a preventive or improvement effect and a method for its production.

### BRIEF DESCRIPTION OF THE DRAWINGS

FIG. 1 is a graph showing the results for Example 3, indicating the effect of arachidonic acid on the spatial recognition of stressed mice.

### BEST MODE FOR CARRYING OUT THE INVENTION

The present invention relates to a preventive or improvement agent for stress-induced brain function impairment and related symptoms or diseases and a composition with a preventive or improvement effect on stress-induced brain function impairment and related symptoms or diseases, comprising as an active ingredient arachidonic acid and/or a compound comprising arachidonic acid as a constituent fatty acid, as well as a method for their production.

As “stress-induced brain function impairment and related symptoms or diseases” there may be mentioned memory and learning ability impairment, emotional disorders (such as depression or melancholia), and the like, but the symptoms and diseases are not limited to these and include all symptoms and diseases associated with stress-induced brain function impairment.

The active ingredient of the invention is arachidonic acid, but any compound comprising arachidonic acid as a constituent fatty acid may be used. As compounds comprising arachidonic acid as a constituent fatty acid there may be mentioned arachidonic acid salts, such as calcium or sodium salts. There may also be mentioned arachidonic acid lower alcohol esters such as arachidonic acid methyl ester and arachidonic acid ethyl ester. There may also be used triglycerides, phospholipids or glycolipids wherein all or a portion of the constituent fatty acid is arachidonic acid. However, the invention is not limited to the compounds mentioned above, and includes any compound comprising arachidonic acid as a constituent fatty acid.

For application to food products, the arachidonic acid is preferably in the form of a triglyceride or phospholipid, and most preferably in the form of a triglyceride. While virtually no natural sources of arachidonic acid-containing triglycerides (i.e., triglycerides including a triglyceride wherein all or a portion of the constituent fatty acid is arachidonic acid) exist, the present inventors have been the first to demonstrate that it is possible to industrially utilize triglycerides comprising arachidonic acid as a constituent fatty acid, that the active ingredients of the invention exhibit apparent behavioral pharmacologic effects in mice subjected to restraint stress and evaluated by a Morris water maze learning test and have preventive or improvement effects for stress-induced brain function impairment and related symptoms or diseases, and that the effects are attributable to arachidonic acid.

According to the invention, therefore, triglycerides including a triglyceride wherein all or a portion of the constituent fatty acid is arachidonic acid (arachidonic acid-containing triglycerides) may be used as the active ingredients of the invention. For application in foods, the arachidonic acid-containing triglycerides are preferably oils or fats (triglycerides) in a form wherein the arachidonic acid content of the total constituent fatty acid of the triglycerides is at least 10 wt % (w/w), more preferably at least 20 wt %, even more preferably at least 30 wt %, and most preferably at least 40 wt %. Thus, the present invention may employ any such compounds which are obtained by culturing microorganisms capable of producing arachidonic acid-containing oils or fats (triglycerides).

As microorganisms capable of producing oils or fats (triglycerides) containing arachidonic acid, there may be mentioned microorganisms belonging to the genera Mortierella, Conidiobolus, Pythium, Phytophthora, Penicillium, Cladosporium, Mucor, Fusarium, Aspergillus, Rhodotorula, Entomophthora, Echinosporangium and Saprolegnia.

As examples of microorganisms belonging to the genus Mortierella, subgenus Mortierella, there may be mentioned Mortierella elongata, Mortierella exigua, Mortierella hygrophila and Mortierella alpina. More specifically, there may be mentioned the strains Mortierella elongata IF08570, Mortierella exigua IF08571, Mortierella hygrophila IF05941, and Mortierella alpina IF08568, ATCC16266, ATCC32221, ATCC42430, CBS219.35, CBS224.37, CBS250.53, CBS343.66, CBS527.72, CBS529.72, CBS608.70, CBS754.68, etc.

All of these strains may be acquired without any special restrictions from the Institute for Fermentation, Osaka (IFO), American Type Culture Collection (ATCC) or Centralbureau voor Schimmelcultures (CBS). There may also be used the strain Mortierella elongata SAM0219 (FERM-P 8703) (deposited under the provisions of the Budapest Treaty on Mar. 19, 1986 with the Patent Microorganism Depository of National Institute of Industrial Science and Technology at Chuo 6, 1-1, Higashi 1-chome, Tsukuba city, Ibaraki pref., Japan, as FERM BP-1239), isolated from soil by the research group for the present invention.

For culturing of a strain to be used for the invention, spores, hypha or a pre-culture solution obtained by pre-culturing the strain may be seeded in a liquid medium or solid medium for culturing. In the case of liquid culturing, the carbon source used may be a common one such as glucose, fructose, xylose, saccharose, maltose, soluble starch, molasses, glycerol or mannitol, although there is no limitation to these.

As nitrogen sources there may be used organic nitrogen sources including urea, and natural nitrogen sources such as peptone, yeast extract, malt extract, meat extract, casamino acid, corn steep liquor, soybean protein, defatted soybean and cotton seed meal, or inorganic nitrogen sources such as sodium nitrate, ammonium nitrate and ammonium sulfate. Trace nutrient sources including inorganic salts such as phosphoric acid salts, magnesium sulfate, iron sulfate and copper sulfate, or vitamins, may also be used if necessary. The medium components are not particularly restricted so long as they are in concentrations which do not prevent growth of the microorganisms. For most practical applications the carbon source may be used at a concentration of 0.1-40 wt % and preferably 1-25 wt %. The initial nitrogen source addition may be at 0.1-10 wt % and preferably 0.1-6 wt %, with optional further feeding of the nitrogen source during culturing.

By controlling the carbon source concentration of the medium it is possible to obtain oils or fats (triglyceride) containing at least 45 wt % arachidonic acid as the active ingredient of the invention. The cell growth phase is the culturing period up to the 2nd-4th day of culturing, while the fat/oil accumulation phase is from the 2nd-4th day of culturing. The initial carbon source concentration is 1-8 wt % and preferably 1-4 wt %, with successive supplemental addition of the carbon source only between the cell growth phase and the early fat/oil accumulation phase, for a total supplemental carbon source addition of 2-20 wt % and preferably 5-15 wt %. The amount of carbon source added between the cell growth phase and the early fat/oil accumulation phase will depend on the initial nitrogen source concentration, and if the carbon source concentration in the medium is 0 from the 7th day of culturing, preferably from the 6th day of culturing and more preferably from the 4th day of culturing, it will be possible to obtain oils or fats (triglyceride) containing at least 45 wt % arachidonic acid, as the active ingredient of the invention.

The culturing temperature for the arachidonic acid-producing cells will differ depending on the microorganism used, but is 5-40° C., preferably 20-30° C., while culturing at 20-30° C. for proliferation of the cells may also be followed by continued culturing at 5-20° C. to produce unsaturated fatty acids. Such temperature control can also be utilized to increase the proportion of polyunsaturated fatty acids among the produced fatty acids. The pH of the medium may be 4-10 and preferably 5-9, for jar fermentor culturing, shake culturing or stationary culturing. The culturing is normally carried out for 2-30 days, preferably 5-20 days and more preferably 5-15 days.

In addition to controlling the carbon source concentration of the medium as a strategy for increasing the proportion of arachidonic acid in the arachidonic acid-containing oils or fats (triglyceride), arachidonic acid-rich oils or fats may also be obtained by selective hydrolysis of the arachidonic acid-containing oils or fats. Since lipases used for such selective hydrolysis do not have specificity for triglycerides and the hydrolytic activity decreases in proportion to the number of double bonds, the ester bonds of the fatty acids other than the polyunsaturated fatty acids are preferentially hydrolyzed. Furthermore, ester-exchange reaction between the produced PUFA glycerides may be used to produce triglycerides with an increased polyunsaturated fatty acid content (“Enhancement of Arachidonic Acid: Selective Hydrolysis of a Single-Cell Oil from Mortierella with Candida cylindracea Lipase”: J. Am. Oil Chem. Soc., 72, 1323, 1998).

Thus, oils or fats (triglyceride) with a high content of arachidonic acid obtained by selective hydrolysis of arachidonic acid-containing oils or fats can be prepared as the active ingredient of the invention. The proportion of arachidonic acid with respect to the total fatty acid content of the arachidonic acid-containing oils or fats (triglyceride) of the invention is preferably higher from the standpoint of eliminating the effect of other fatty acids, but it does not necessarily have to be a high proportion, and in fact the absolute amount of arachidonic acid can pose a problem for application to some foods. Oils or fats (triglycerides) containing arachidonic acid at 10 wt % or greater can be suitably used in most cases.

As triglycerides wherein all or a portion of the constituent fatty acid is arachidonic acid according to the invention, there may be used triglycerides having medium chain fatty acids bonded at the 1,3-positions and arachidonic acid bonded at the 2-position. The oils or fats (triglycerides) used may also comprise at least 5 mole percent, preferably at least 10 mole percent, more preferably at least 20 mole percent and most preferably at least 30 mole percent, of triglycerides having medium chain fatty acids bonded at the 1,3-positions and arachidonic acid bonded at the 2-position. The medium chain fatty acids bonded at the 1,3-positions of the triglyceride may be selected from among C6-12 fatty acids. As examples of C6-12 fatty acids there may be mentioned caprylic acid or capric acid, with 1,3-capryloyl-2-arachidonoyl-glycerol (hereinafter, “8A8”) being particularly preferred.

Such triglycerides having medium chain fatty acids bonded at the 1,3-positions and arachidonic acid bonded at the 2-position are optimum oils or fats (triglycerides) for elderly persons. Generally speaking, ingested oils or fats (triglycerides) are hydrolyzed by pancreatic lipases upon entering the small intestine, but since pancreatic lipases are 1,3-specific, the 1,3-positions of the triglycerides are cleaved to form two free fatty acids while simultaneously producing a single 2-monoacylglycerol (MG). As 2-MG has extremely high bile solubility and is highly absorbable, the 2-position fatty acid is generally considered to be better absorbed. In addition, 2-MG dissolved in bile acid acts as a surfactant and thus increases the absorption of the free fatty acids.

The free fatty acids and 2-MG then form bile acid complex micelles together with cholesterol, phospholipids and the like and are incorporated into the intestinal epithelial cells where triacylglycerols are resynthesized, being finally released into the lymph as chylomicrons. However, the fatty acid specificity of pancreatic lipases is higher for saturated fatty acids, whereas arachidonic acid is not as easily cleaved. Another problem is that pancreatic lipase activity declines with age, and therefore triglycerides having medium chain fatty acids bonded at the 1,3-positions and arachidonic acid bonded at the 2-position are more optimal oils or fats (triglycerides) for the elderly.

One specific production method for triglycerides having medium chain fatty acids bonded at the 1,3-positions and arachidonic acid bonded at the 2-position is a method using a lipase which acts only on the 1,3-position ester bonds of triglycerides, in the presence of arachidonic acid-containing oils or fats (triglyceride) and a medium chain fatty acid.

The oils or fats (triglyceride) starting material are a triglyceride comprising arachidonic acid as a constituent fatty acid, but in the case of a high proportion of arachidonic acid with respect to the total constituent fatty acid of the triglycerides, reduced reaction yield due to excess unreacted oils or fats (the triglyceride starting material and triglycerides wherein only one of the 1,3-position fatty acids has been converted to a medium chain fatty acid) can be prevented if the temperature is above the normal enzyme reaction temperature of 20-30° C., such as 30-50° C. and preferably 40-50° C.

As examples of lipases which act specifically on the 1,3-position ester bonds of triglycerides there may be mentioned lipases produced by microorganisms such as Rhizopus, Rhizomucor and Aspergillus, as well as porcine pancreatic lipases. Any such commercially available lipases may be used. For example, there may be mentioned Rhizopus delemar lipase (Talipase, Tanabe Pharmaceutical Co., Ltd.), Rhizomucor miehei lipase (Ribozyme IM, Novo Nordisk Co., Ltd.) and Aspergillus niger lipase (Lipase A, Amano Pharmaceutical Co., Ltd.), although there is no limitation to these enzymes and any 1,3-specific lipases may be used.

The form of the lipase used is preferably an immobilized form on an immobilizing support in order to impart heat resistance to the enzyme, since the reaction temperature is 30° C. or above and preferably 40° C. or above for increased reaction efficiency. The immobilizing support may be a porous (highly porous) resin, for example, an ion-exchange resin with pores of approximately 100 Å or greater such as Dowex MARATHON WBA. However, this condition is not restrictive on the immobilizing support, and any immobilizing support capable of imparting heat resistance may be used.

The immobilizing support may be suspended in an aqueous solution of a 1,3-specific lipase at a weight proportion of 0.5-20 of the latter with respect to the former, and a 2- to 5-fold amount of cold acetone (for example, −80° C.) may be slowly added to the suspension while stirring to form a precipitate. The precipitate may then be dried under reduced pressure to prepare the immobilized enzyme. As a simpler method, a 1,3-specific lipase in a proportion of 0.05-0.4 with respect to the immobilizing support may be dissolved in a minimal amount of water and mixed with the immobilizing support while stirring and dried under reduced pressure to prepare the immobilized enzyme. This procedure can immobilize approximately 90% lipase on the support, but since absolutely no ester exchange activity will be exhibited in that state, pretreatment may be carried out in a substrate containing 1-10 wt % (w/v) water and preferably a substrate containing 1-3 wt % water, in order to activate the immobilized enzyme to maximum efficiency before it is provided for production.

The amount of water added to the reaction system is extremely important depending on the type of enzyme, because a lack of water will impede ester exchange while an excess of water will cause hydrolysis and a reduced glyceride yield (since hydrolysis will produce diglycerides and monoglycerides) . However, if the immobilized enzyme used has been activated by pretreatment the amount of water added to the reaction system is no longer crucial, and an efficient ester exchange reaction can be carried out even in a completely water-free system. Also, selection of the type of enzyme agent may allow the pretreatment step to be omitted.

Thus, by using a heat-resistant immobilized enzyme and raising the enzyme reaction temperature, it is possible to efficiently produce triglycerides having medium chain fatty acids bonded at the 1,3-positions and arachidonic acid bonded at the 2-position (8A8), without lowering the reaction efficiency even for arachidonic acid-containing oils or fats (triglycerides) with low reactivity for 1,3-specific lipases.

A method for production of a dietary product having a preventive or improvement effect on stress-induced brain function impairment and related symptoms or diseases, involves adding arachidonic acid and/or a compound including arachidonic acid as a constituent fatty acid alone, or in combination with a dietary material containing substantially no arachidonic acid or only a slight amount thereof. Here, a “slight amount” means that even if arachidonic acid is present in the dietary product material and a food composition containing it is ingested by a human, the amount does not reach the daily amount of arachidonic acid consumption according to the invention, as described hereunder.

An unlimited number of uses exist for oils or fats (triglycerides) wherein all or a portion of the constituent fatty acid is arachidonic acid: for example, they may be used as starting materials and additives for foods, beverages, cosmetics and pharmaceuticals. The purposes of use and amounts of use are also completely unrestricted.

As examples of food compositions there may be mentioned ordinary foods, as well as functional foods, nutritional supplements, food for specified health uses, preterm infant formula, term infant formula, infant foods, maternal foods or geriatric foods. As examples of fat/oil-containing foods there may be mentioned natural fat/oil-containing foods such as meat, fish and nuts, foods to which oils or fats are added during preparation, such as soups, foods employing oils or fats as heating media, such as donuts, oils or fats foods such as butter, processed foods to which oils or fats are added during processing, such as cookies, or foods which are sprayed or coated with oils or fats upon finishing, such as hard biscuits. Such compositions may also be added to agricultural foods, fermented foods, livestock feeds, marine foods and beverages which contain no oils or fats. They may also be in the form of functional foods or pharmaceuticals, and for example, in processed form such as enteral nutrients, powders, granules, lozenges, oral solutions, suspensions, emulsions, syrups and the like.

A composition of the invention may also contain various carriers or additives ordinarily used in foods and beverages, pharmaceuticals or quasi drugs, in addition to the active ingredient of the invention. Antioxidants are particularly preferred as additives to prevent oxidation of the active ingredient of the invention. As examples of antioxidants there may be mentioned natural antioxidants such as tocopherols, flavone derivatives, phyllodulcins, kojic acid, gallic acid derivatives, catechins, fukiic acid, gossypol, pyrazine derivatives, sesamol, guaiaol, guaiac acid, p-coumaric acid, nordihydroguaiaretic acid, sterols, terpenes, nucleotide bases, carotenoids, lignans and the like, and synthetic antioxidants including ascorbic palmitic acid esters, ascorbic stearic acid esters, butylhydroxyanisole (BHA), butylhydroxytoluene (BHT), mono-t-butylhydroquinone (TBHQ) and 4-hydroxymethyl-2,6-di-t-butylphenol (HMBP).

As tocopherols there may be mentioned α-tocopherol, β-tocopherol, γ-tocopherol, δ-tocopherol, ε-tocopherol, ζ-tocopherol, η-tocopherol and tocopherol esters (tocopherol acetate and the like), as well as tocopherol analogs. As examples of carotenoids there may be mentioned β-carotene, cantaxanthine, astaxanthine and the like.

The composition of the invention may also contain, in addition to the active ingredient of the invention, supports such as carrier supports, extenders, diluents, bulking agents, dispersing agents, excipients, binder solvents (for example, water, ethanol and vegetable oils), dissolving aids, buffering agents, dissolving accelerators, gelling agents, suspending agents, wheat flour, rice flour, starch, corn starch, polysaccharides, milk protein, collagen, rice oil, lecithin and the like. As examples of additives there may be mentioned vitamins, sweeteners, organic acids, coloring agents, aromatic agents, moisture-preventing agents, fibers, electrolytes, minerals, nutrients, antioxidants, preservatives, fragrances, humectants, natural food extracts, vegetable extracts and the like, although there is no limitation to these.

Arachidonic acid is the main active ingredient of the compound which is either arachidonic acid or comprises arachidonic acid as a constituent fatty acid. The daily intake of arachidonic acid from dietary sources has been reported to be 0.14 g in the Kanto region and 0.19-0.20 g in the Kansai region of Japan (Shishitsu Eiyougaku 4, 73, 1995), and in consideration of reduced oils or fats intake and reduced pancreatic lipase function in the elderly, a correspondingly greater amount of arachidonic acid must be ingested. Thus, the daily intake of the arachidonic acid or the compound comprising arachidonic acid as a constituent fatty acid according to the invention for an adult (for example, 60 kg body weight) is 0.001-20 g, preferably 0.01-10 g, more preferably 0.05-5 g and most preferably 0.1-2 g, based on the arachidonic acid content.

When the active ingredient of the invention is to be actually applied for a food or beverage product, the absolute amount of arachidonic acid in the product is an important factor. However, since the absolute amount added to foods and beverages will differ depending on the amount of consumption of those foods or beverages, triglycerides including a triglyceride wherein all or a portion of the constituent fatty acid is arachidonic acid may be added to food products in amounts of at least 0.001 wt %, preferably at least 0.01 wt % and more preferably at least 0.1 wt % in terms of arachidonic acid. For addition to food and beverage products of triglycerides having medium chain fatty acids bonded at the 1,3-positions and arachidonic acid bonded at the 2-position, the amount may be at least 0.0003 wt %, preferably at least 0.003 wt % and more preferably at least 0.03 wt %.

When the composition of the invention is to be used as a pharmaceutical, it may be produced according to a common method in the field of pharmaceutical preparation techniques, such as according to a method described in the Japanese Pharmacopeia or a similar method.

When the composition of the invention is to be used as a pharmaceutical, the content of the active ingredient in the composition is not particularly restricted so long as the object of the invention is achieved, and any appropriate content may be employed.

When the composition of the invention is to be used as a pharmaceutical, it is preferably administered in the form of an administrable unit, and especially in oral form. The dosage of the composition of the invention will differ depending on age, body weight, symptoms and frequency of administration, but for example, the arachidonic acid and/or compound including arachidonic acid as a constituent fatty acid according to the invention may be administered at about 0.001-20 g, preferably 0.01-10 g, more preferably 0.05-5 g and most preferably 0.1-2 g (as arachidonic acid) per day for adults (approximately 60 kg), either once a day or divided among multiple doses, such as three separate doses.

The major fatty acid components of phospholipid membranes in the brain are arachidonic acid and docosahexaenoic acid, and therefore from the standpoint of balance, a combination with docosahexaenoic acid is preferred. Also, since the proportion of eicosapentaenoic acid in brain phospholipid membranes is very small, a combination of arachidonic acid and docosahexaenoic acid containing virtually no eicosapentaenoic acid is especially preferred. Furthermore, the arachidonic acid/docosahexaenoic acid ratio in the combination of the arachidonic acid and docosahexaenoic acid is preferably in the range of 0.1-15, and more preferably in the range of 0.25-10. Also, the amount of eicosapentaenoic acid in the food or beverage preferably does not exceed ⅕ of the arachidonic acid (weight ratio).

### EXAMPLES

The present invention will now be explained in greater detail by the following examples, with the understanding that the invention is not limited to these examples.

### Example 1

### Method for Production of arachidonic acid-Containing triglycerides

Mortierella alpina CBS754.68 was used as the arachidonic acid-producing strain. After preparing 6 kL of medium containing 1.8% glucose, 3.1% defatted soybean powder, 0.1% soybean oil, 0.3% KH₂PO₄, 0.1% Na₂SO₄, 0.05% CaCl₂.2H₂O and 0.05% MgCl₂.6H₂O in a 10 kL culturing tank, the initial pH was adjusted to 6.0. A 30 L portion of the preculturing solution was transferred for 8 days of jar fermentor culturing under conditions with a temperature of 26° C., an airflow of 360 m³/h and an internal pressure of 200 kPa. The stirring rate was adjusted to maintain a dissolved oxygen concentration of 10-15 ppm. Also, the glucose concentration was adjusted by the feeding culture method for a glucose concentration in the range of 1-2.5% in the medium up to the 4th day, with 0.5-1% maintained thereafter (where the percentage values are weight (W/V)%).

After completion of the culturing, the cells containing triglycerides having arachidonic acid as a constituent fatty acid were collected by filtration and drying, and the oils or fats portion was extracted from the collected cells by hexane extraction and subjected to dietary oils or fats purification steps (degaussing, deoxidation, deodorization, decolorizing) to obtain 150 kg of arachidonic acid-containing triglycerides (triglycerides including a triglyceride wherein all or a portion of the constituent fatty acid is arachidonic acid). The obtained oils or fats (triglycerides) were methylesterified, and the obtained fatty acid methyl ester mixture was analyzed by gas chromatography and found to have an arachidonic acid proportion of 40.84 wt % of the total fatty acid.

The contents of palmitic acid, stearic acid, oleic acid, linoleic acid, γy-linolenic acid and dihomo-γ-linolenic acid were 11.63%, 7.45%, 7.73%, 9.14%, 2.23% and 3.27% by weight, respectively. The arachidonic acid-containing oils or fats (triglycerides) (TGA40S) were also ethylesterified, and the fatty acid ethyl ester mixture including 40 wt % arachidonic acid ethyl ester was separated and purified by an established high-performance liquid chromatography method to obtain 99 wt % arachidonic acid ethyl ester.

### Example 2

### Production of triglycerides Including at least 5 Mole Percent 8A8

After suspending 100 g of an ion-exchange resin carrier (Dowex MARATHON WBA: Dow Chemical) in 80 ml of Rhizopus delemar lipase aqueous solution (12.5% Talipase powder, Tanabe Pharmaceutical Co., Ltd.), 240 ml of cold acetone (−80° C.) was stirred therewith and the mixture was dried under reduced pressure to obtain the immobilized lipase.

Next, 80 g of the triglycerides containing 40 wt % arachidonic acid (TGA40S) obtained in Example 1, 160 g of caprylic acid, 12 g of the aforementioned immobilized lipase and 4.8 ml of water were reacted for 48 hours at 30° C. while stirring (130 rpm). Upon completion of the reaction, the reaction solution was removed to obtain the activated immobilized enzyme.

A 10 g portion of immobilized lipase (Rhizopus delemar lipase, carrier: Dowex MARATHON WBA) was then packed into a jacketed glass column (1.8×12.5 cm, 31.8 ml volume), and the reaction oils or fats comprising a mixture of the TGA40S obtained in Example 1 and caprylic acid (TGA40S: caprylic acid=1:2) was flowed through the column at a fixed speed (4 ml/h) for continuous reaction, to obtain 400 g of reaction oils or fats. The column temperature was 40-41° C. The unreacted caprylic acid and free fatty acids were removed from the obtained reaction oils or fats by molecular distillation, and then subjected to dietary oils or fats purification steps (degumming, deoxidation, deodorization, decolorizing) to obtain 8A8-containing oils or fats (triglycerides).

The 8A8 proportion of the obtained 8A8-containing oils or fats (triglycerides) was determined by gas chromatography and high-performance liquid chromatography to be 31.6 mole percent. (Incidentally, the proportions of 8P8, 8O8, 8L8, 8G8 and 8D8 were 0.6, 7.9, 15.1, 5.2 and 4.8 mole percent, respectively. The fatty acids P, O, L, G and D bonded at the triglyceride 2-position represent palmitic acid, oleic acid, linoleic acid, γ-linolenic acid and dihomo-γ-linolenic acid, respectively, and therefore 8P8 represents 1,3-capryloyl-2-palmitolein-glycerol, 8O8 represents 1,3-capryloyl-2-oleoyl-glycerol, 8L8 represents 1,3-capryloyl-2-linoleoyl-glycerol, 8G8 represents 1,3-capryloyl-2-γ-linolenoyl-glycerol and 8D8 represents 1,3-capryloyl-2-dihomo-γ-linolenoyl-glycerol). Separation and purification from the obtained 8A8-containing oils or fats (triglycerides) by an established high-performance liquid chromatography method yielded 96 mole percent 8A8.

### Example 3

### Evaluation of Learning Ability Effect of TGA40S by Morris Water Maze Learning Test

The experimental groups consisted of 56 two- to three-month-old male ICR mice, divided into a control diet group (27 mice) and a TGA40S-containing diet group (29 mice), with the control diet or TGA40S-containing diet shown in Table 1 being given to each group for 3 weeks. Each group was further divided into non-restrained groups (non-restrained control diet group (13), non-restrained arachidonic acid (ARA) diet group (15)) and restrained groups (restrained control diet group (14), restrained ARA diet group (14)). The restraining was accomplished using a wire mesh restraining tube, once for a 6 hour period three weeks after the start of feeding. The control diet or TGA40S-containing diet shown in Table 1 continued to be fed to each group for the remaining experiment period. The TGA40S used for the TGA40S-containing diet was the product obtained in Example 1.

TABLE 1
Experimental diet

| | Control diet | TGA40S-added diet |
|---|---|---|
| Casein (g/kg) | 200 | 200 |
| DL-methionine | 3 | 3 |
| Corn starch | 150 | 150 |
| Sucrose | 500 | 500 |
| Cellulose powder | 50 | 50 |
| Corn oil | 50 | 45 |
| Mineral AIN-76 | 35 | 35 |
| Vitamin AIN-76 | 10 | 10 |
| Choline bitartrate | 2 | 2 |
| Vitamin E | 0.05 | 0.05 |
| TGA40S | 0 | 5 |

Since the daily ingestion was approximately 5 g per mouse, the daily intake of TGA40S was 25 mg per mouse. Also, since the total fatty acids bonded to the arachidonic acid-containing oils or fats (triglycerides) prepared in Example 1 included 40 wt % arachidonic acid, the daily intake of arachidonic acid was 10 mg per mouse.

The 6-hour restraint with a wire mesh restraining tube was immediately followed by a Morris water maze learning test. The Morris water maze learning test is widely used in Europe and the U.S., and is conducted by pouring water blackened with India ink into a water tank (100 cm diameter, 35 cm height) (liquid surface height: 20 cm), setting therein an escape platform of just a size to allow a mouse to stand (the escape platform is submerged and invisible to a mouse swimming in the water tank), and then placing the mouse subject at a prescribed location of the water tank (starting point), forcing it to swim to the escape platform, in order to test its learning ability based on spatial recognition which is associated with the memory-governing hippocampus.

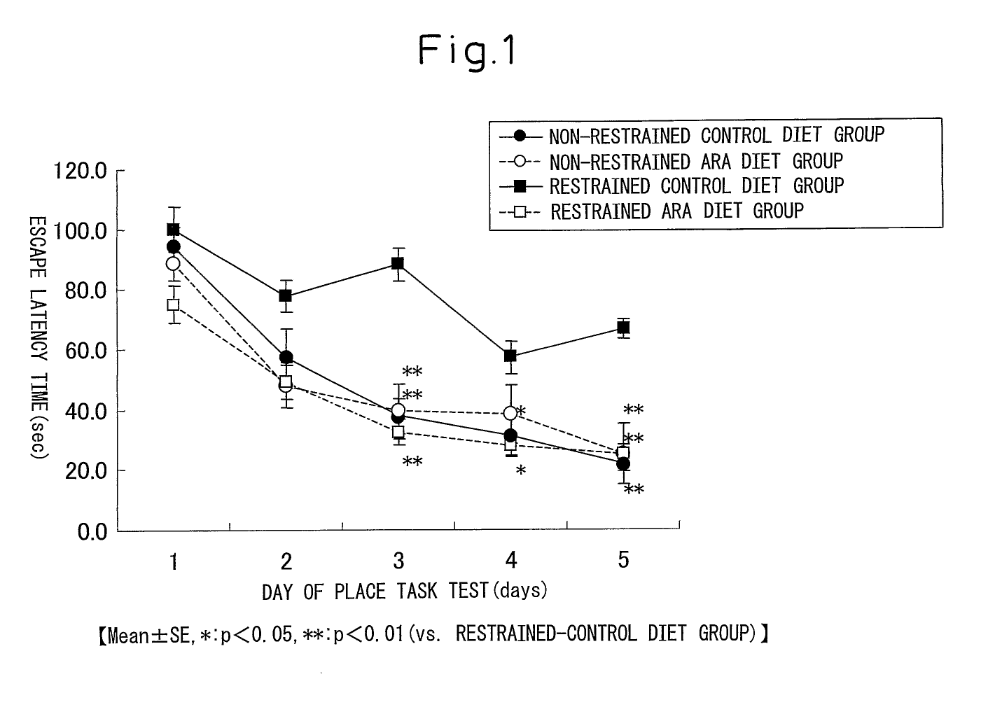

The water temperature was 30° C.±1° C., each trial was limited to 120 seconds with an interval of 60 seconds between trials, and five trials were conducted each day for 5 days. The time required for the mouse to reach the escape platform (escape latency time) was recorded as the learning index. No difference was observed between the control diet mice and ARA diet mice in the absence of restraint stress. However, the mice of the control diet group which had experienced restraint stress clearly exhibited reduced learning ability compared to the non-restrained mice, whereas mice given TAG40S (arachidonic acid) exhibited the same level of learning ability as the mice without restraint stress (FIG. 1).

Thus, for the first time it has been clearly demonstrated that administration of TGA40S improves learning ability or cognitive ability which has declined as a result of stress, and that arachidonic acid exhibits an improving effect against decline in learning ability or cognitive ability as a result of stress.

### Example 4

### Preparation of Capsules Comprising arachidonic acid-Containing Oils or Fats (triglycerides)

Water was added to 100 parts by weight of gelatin and 35 parts by weight of food additive grade glycerin for dissolution at 50-60° C., to prepare a gelatin coating with a viscosity of 2000 cp. Next, 0.05 wt % of vitamin E oil was combined with the arachidonic acid-containing oils or fats (triglycerides) obtained in Example 1 to prepare filling 1. Vitamin E was also added to oils or fats (triglycerides) containing 32 mole percent of the 8A8 obtained in Example 2 to prepare filling 2. Also, 50 wt % of the arachidonic acid-containing oils or fats (triglycerides) obtained in Example 1 was combined with 50 wt % fish oil (tuna oil: the eicosapentaenoic acid and docosahexaenoic acid proportions of the total fatty acids were 5.1% and 26.5%, respectively) and then 0.05 wt % vitamin E oil was added to prepare filling 3.

Also, 80 wt % of the arachidonic acid-containing oils or fats (triglycerides) obtained in Example 1 was combined with 20 wt % fish oil (tuna oil: the eicosapentaenoic acid and docosahexaenoic acid proportions of the total fatty acids were 5.1% and 26.5%, respectively) and then 0.05 wt % vitamin E oil was added to prepare filling 4. Separately, 0.05 wt % of vitamin E oil was combined with the 99% arachidonic acid ethyl ester obtained in Example 1 to prepare filling 5. These fillings 1 to 5 were used for production of soft capsules containing 180 mg of filling per capsule, obtained by capsule molding and drying by ordinary methods.

### Example 5

### Use for Oil Infusion

After combining 400 g of the oils or fats (triglycerides) containing 96 mole percent 8A8 obtained in Example 2, 48 g of purified egg yolk lecithin, 20 g of oleic acid, 100 g of glycerin and 40 ml of 0.1 N caustic soda and dispersing the mixture with a homogenizer, distilled water for injection was added to make 4 liters. This was emulsified with a high-pressure spray emulsifier to prepare a lipid emulsion. The lipid emulsion was dispensed into plastic bags at 200 ml per bag and then subjected to high-pressure steam sterilization treatment at 121° C. for 20 minutes to prepare an oil infusion.

### Example 6

### Use for Juice

A 2 g portion of β-cyclodextrin was added to 20 ml of 20% aqueous ethanol, and then 100 mg of the arachidonic acid-containing triglycerides obtained in Example 1 (containing 0.05% vitamin E) were added thereto while stirring with a stirrer, and the mixture was incubated for 2 hours at 50° C. After room temperature cooling (approximately 1 hour), stirring was continued while incubating for 10 hours at 4° C. The resulting precipitate was recovered by centrifugal separation and then washed with n-hexane and lyophilized to obtain 1.8 g of a cyclodextrin clathrate compound comprising arachidonic acid-containing triglycerides. A 1 g portion of this powder was uniformly mixed into 10 L of juice to prepare a juice comprising arachidonic acid-containing triglycerides.

1. A composition with a preventive or improvement effect on stress-induced brain function impairment and related symptoms or diseases, comprising arachidonic acid and/or a compound comprising arachidonic acid as a constituent fatty acid.

2. A composition according to claim 1, wherein said compound comprising arachidonic acid as a constituent fatty acid is an arachidonic acid alcohol ester, or a triglyceride, phospholipid or glycolipid wherein all or a portion of the constituent fatty acid is arachidonic acid.

3. A composition according to claim 2, wherein the triglyceride in which all or a portion of the constituent fatty acid is arachidonic acid is a triglyceride having medium chain fatty acids bonded at the 1,3-positions and arachidonic acid bonded at the 2-position.

4. A composition according to claim 3, wherein said medium chain fatty acids are selected from among C6-12 fatty acids.

5. A composition with a preventive or improvement effect on stress-induced brain function impairment and related symptoms or diseases, comprising triglycerides which include a triglyceride in which all or a portion of the constituent fatty acid is arachidonic acid.

6. A composition according to claim 5, characterized in that the arachidonic acid content of said triglycerides which include a triglyceride in which all or a portion of the constituent fatty acid is arachidonic acid, is at least 10 wt % of the total fatty acids of the triglycerides.

7. A composition according to claim 5, wherein said triglycerides which include a triglyceride in which all or a portion of the constituent fatty acid is arachidonic acid, are extracted from a microorganism belonging to the genus Mortierella, Conidiobolus, Pythium, Phytophthora, Penicillium, Cladosporium, Mucor, Fusarium, Aspergillus, Rhodotorula, Entomophthora, Echinosporangium or Saprolegnia.

8. A composition according to claim 5, wherein said triglycerides which include a triglyceride in which all or a portion of the constituent fatty acid is arachidonic acid, are triglycerides containing virtually no eicosapentaenoic acid.

9. A composition with a preventive or improvement effect on stress-induced brain function impairment and related symptoms or diseases, comprising triglycerides of which at least 5 mole percent consists of a triglyceride having medium chain fatty acids bonded at the 1,3-positions and arachidonic acid bonded at the 2-position.

10. A composition according to claim 9, wherein said medium chain fatty acids are selected from among C6-12 fatty acids.

11. A composition according to claim 1, wherein said symptoms related to stress-induced brain function impairment include memory and learning ability impairment.

12. A composition according to claim 1, wherein said symptoms related to stress-induced brain function impairment include cognitive ability impairment.

13. A composition according to claim 1, wherein said symptoms related to stress-induced brain function impairment include depression.

14. A composition according to claim 1, wherein said diseases related to stress-induced brain function impairment include melancholia.

15. A composition according to claim 1, wherein said composition is a food composition or pharmaceutical composition.

16. A composition according to claim 15, characterized in that said food composition is a common food (food and drink), functional food, nutritional supplement, food for specified health uses, preterm infant formula, term infant formula, infant food, maternal food or geriatric food.

17. A composition according to claim 1, which comprises docosahexaenoic acid and/or a compound comprising docosahexaenoic acid as a constituent fatty acid.

18. A composition according to claim 17, wherein said compound comprising docosahexaenoic acid as a constituent fatty acid is a docosahexaenoic acid alcohol ester, or a triglyceride, phospholipid or glycolipid wherein all or a portion of the constituent fatty acid is docosahexaenoic acid.

19. A composition according to claim 17, characterized in that the arachidonic acid/docosahexaenoic acid ratio (by weight) in the combination of said arachidonic acid and docosahexaenoic acid is in the range of 0.1 to 15.

20. A composition according to claim 1, characterized in that the amount of eicosapentaenoic acid in the composition does not exceed ⅕ of the arachidonic acid in the composition.

21. A method for production of a dietary product having a preventive or improvement effect on stress-induced brain function impairment and related symptoms or diseases, the method being characterized by adding arachidonic acid and/or a compound comprising arachidonic acid as a constituent fatty acid alone, or in combination with a dietary material containing substantially no arachidonic acid or only a slight amount thereof.

22. A method for prevention or medical treatment of stress-induced brain function impairment and related symptoms or diseases, which comprises administering arachidonic acid and/or a compound comprising arachidonic acid as a constituent fatty acid, to a patient in need of its administration.
